(19) United States
(12) Patent Application Publication
(10) Pub. No.: US 2022/0280733 A1
(43) Pub. Date: Sep. 8, 2022

(54) Delivery Apparatus, Method And System for Inhalable Medications

(71) Applicant: Kathryn L. Brown, Mead, WA (US)

(72) Inventor: Kathryn L. Brown, Mead, WA (US)

(21) Appl. No.: 17/470,400

(22) Filed: Sep. 9, 2021

Related U.S. Application Data
(60) Provisional application No. 63/157,585, filed on Mar. 5, 2021.

(51) Int. Cl. A61M 15/00; B65D 75/30

(52) U.S. Cl. CPC A61M 15/0061; B65D 75/305; A61M 2209/06; A61M 2202/048

(57) ABSTRACT

Adherable, perforated patch worn inside or outside a facial mask delivering an inhalable product to airspace proximate to human respiratory airway. A disposable cover and disposable seal is removed, the patch is applied to inside or outside of a mask, with the mask worn over the mouth and nose. The patch delivers inhalable product to air proximate to the patch. Disposable cover, seal and tamper proof wrapper. Methods of manufacturing, distribution and utilization of the patch are disclosed.

### CROSS REFERENCE TO RELATED APPLICATIONS

The present application claims priority in and to U.S. patent application 63/157,585 filed Mar. 5, 2021, and further is incorporated herein by reference.

### TECHNICAL FIELD

The presently disclosed technology relates to a vehicle for delivering inhalable medication or other chemical substance via a perforated patch. Apparatus and methods relating to an adherable patch, and related mask apparatus or attachment structures, will be disclosed.

### BACKGROUND

Existing technologies used by patients to inhale medicines or medications rely principally on a breathing apparatus such as a nebulizer, inhaler or other complex machine which converts a medication or inhalable substance into airborne particles in the air. Unfortunately, all of these devices require either electricity or compressed air to distribute such medicines or inhalable substances.

A nebulizer requires significant energy and equipment to work properly, namely a machine to create positive air pressure to combine the air with the medication or inhalable substance. This task requires a form of energy, which is typically electricity. Thereafter, the mixed air and medication/substance must be introduced to the nose and/or mouth of the patient or consumer inhaling the product, such as through tubing to a wearable face mask. The tubing can be a nuisance as well as reducing the efficiency of the application of the inhaled product, since the tubing itself becomes coated with the inhalable product.

An inhaler is also a relatively complex machine, in that a sealed container of one or more compressed gases must be manufactured, quality controlled and distributed along the commerce chain. Thereafter, the device must safely and reliably utilize the compressed air. Unfortunately, this typically results in a relatively uncontrolled application of the inhaled product.

What is needed in the present art is a simple, reliable and accurate vehicle for distribution and delivery of an inhalable product (e.g. medication or other inhalable substance in a controlled dosage). Preferably, such a vehicle does not require power, moving parts, compressed gas or other complex apparatus.

In present times, with the onset of the Covid-19 virus and its variants, a significant portion of the population in civilized countries avails itself to wearing facial masks. In present times, I have also utilized such a mask (e.g. ordinary surgical/facial mask) to reduce the likelihood of both catching or causing the spread of such a virus.

Due to my health, I also utilize inhalable products, which I have found are very difficult to inhale while wearing such a mask. Therefore, to overcome this problem, I have developed a breakthrough advanced apparatus, method and system for distributing and using inhalable products.

### SUMMARY

I have developed an adherable, perforated patch that can be worn inside or outside of a facial mask, that can deliver inhalable products to the wearer of the mask without power, moving parts, compressed air or other complex devices.

The present disclosure describes apparatus directed to an adherable patch impregnated with an inhalable product, preferably packaged in a disposable tamper proof wrapper. To use, the wrapper of the patch is removed, a disposable cover over the adhesive material is removed, the patch is applied to a facial mask, and the mask worn over the mouth and/or nose. Once the seal is broken, the patch delivers the impregnated inhalable product to the airspace proximate to both the patch and respiratory airways (e.g. to the mouth and/or nose).

In one aspect, embodiments of the adherable patch are applied to the outside of a mask, wherein the inhalable product travels through a facial mask into the airways of the nose and mouth.

In another aspect, embodiments of the adherable patch are applied to the inside of a facial mask, wherein the inhalable product travels directly into the respiratory airways of the nose and mouth.

In other aspects, embodiments of the patch in various materials, adhesion structures and sizes/shapes are disclosed, as well as a system comprising of the adherable patch and an accompanying facial mask.

Methods of manufacturing, distributing and utilizing the patch are also disclosed.

### BRIEF DESCRIPTION OF THE DRAWINGS

Embodiments of the invention are illustrated by way of example, and not by way of limitation, in the figures of the accompanying drawings and in which like reference numerals refer to similar elements, wherein:

FIG. 1 is an environmental front view of an embodiment of an adherable, perforated patch applied to the outside of a facial mask.

FIG. 2 is an environmental side view of the embodiment of an adherable, perforated patch applied to the outside of a facial mask of FIG. 1.

FIG. 3 is a front view of the embodiment of an adherable, perforated patch in FIGS. 1 & 2.

FIG. 4 is a back view of the embodiment of an adherable, perforated patch in FIGS. 1-3.

FIG. 5 is a cross-section side view of the embodiment of an adherable, perforated patch in FIGS. 1-4.

FIG. 6 is an environmental front view of an embodiment of an adherable, perforated patch applied to the inside of a facial mask.

FIG. 7 is an environmental side view of the embodiment of an adherable, perforated patch applied to the inside of a facial mask of FIG. 6.

FIG. 8 is a front view of the embodiment of an adherable, perforated patch in FIGS. 6 & 7.

FIG. 9 is a back view of the embodiment of an adherable, perforated patch in FIGS. 6-8.

FIG. 10 is a cross-section side view of the embodiment of an adherable, perforated patch in FIGS. 6-9.

FIG. 11 is a side view of the embodiment of an adherable, perforated patch intended to be worn outside of a mask, configured with a disposable cover and a disposable seal.

FIG. 12 is a side view of the embodiment of an adherable, perforated patch intended to be worn inside of a mask, configured with a disposable cover and a disposable seal.

FIG. 13 is a flow chart of an embodiment of a method of use of an adherable patch worn on the outside of a facial mask.

FIG. 14 is a flow chart of an embodiment of a method of use of an adherable patch worn on the inside of a facial mask.

FIG. 15 is a flow chart of an embodiment of a method of manufacture of an adherable patch.

FIG. 16 is a flow chart of an embodiment of a method of procurement of an adherable patch.

FIG. 17 is a flow chart of an embodiment of a method of procurement of a system for delivery of an inhalable product.

### DETAILED DESCRIPTION

In the following description, for the purposes of explanation, specific details are set forth in order to provide a thorough understanding of the invention. However, it will be apparent that the invention may be practiced without these specific details. In other instances, well-known structures and devices may be depicted in block diagram form or simplified form in order to avoid unnecessary obscuring of the invention. Section titles and references appearing within the following paragraphs are intended for the convenience of the reader and should not be interpreted to restrict the scope of the information presented at any given location. As such, various aspects and features of example embodiments of the invention are described in more detail hereinafter in the following sections.

### SUMMARY

I have invented a simpler, more cost-effective option for various medicines, medications or other inhaled products, to be inhaled, as follows. I have invented an adherable, perforated patch, which can also be known as an adherable patch or a perforated patch, or simply as a “patch” herein.

Any number of inhalable products can be applied to or impregnated within my patch. The patch is then adhered to a facial mask (e.g. surgical mask) or other face covering (e.g. facial scarf, tube facial wrap, etc.), either on the outside or the inside of the facial mask or facial covering.

The adherable patch is preferably a permeable patch that allows air to pass through it, serving as a convenient vehicle to allow the inhalable product to naturally evaporate, aerosolize or otherwise become airborne, thereafter inhaled by a human wearing the patch via a facial mask or face covering. The device does not require power, compressed air nor complex mechanical or chemical mechanisms.

Such an adherable patch or adhesive patch may be any convenient physical form that allows a product to be impregnated within the patch or applied onto the patch. The patch can be transparent or opaque, or therebetween, and may comprise of fabric, foam, cotton, mesh or any other material capable of holding an inhalable product (e.g. medicine, non-medication, essential oil, etc.) to be inhaled. The patch can be made in any size appropriate to adhere to a facial mask or facial covering. Alternatively, the patch can be held in place by the facial mask or facial covering rather than by adhesive (e.g. via a pocket in the facial mask).

It is preferable, but not essential, that the patch comprise some form of adhesive such that it can be applied directly to a facial mask or face covering being worn. The adherable patch is preferably surrounded by a light strength adhesive, easily removed without damage to the mask or facial covering. Such an adhesive function can be performed by conventional adhesive used in first aid bandages, or loop and hook fabric (e.g. Velcro), snap or other conventional attachment means.

In other embodiments, a pocket can be fabricated in the facial mask or facial covering that allows a patch with or without adhesive material to be placed inside of it.

In yet other embodiments, the facial mask or facial covering can comprise simply a facial covering such as a bandana or scarf which is configured on the face.

The patch itself is preferably breathable, thereby allowing air to pass through it, such that a small amount of the medicine and medication is transferred into the same air that is to be inhaled by the patient.

It is the express intention that the facial mask or facial covering be configured over the nose and/or the mouth, such that the human respiratory airways (e.g. nose and mouth) are partially or wholly covered by the facial mask or facial covering. In this manner, the airspace proximate to the human respiratory airway, near the patch, is inhaled.

The patch is intended as a vehicle or physical means for a product to be dispensed and may be configured to hold an appropriate dosage of medicine or medication, or other inhalable product, and may be configured to be manufactured or sold in different dosages, periods of use, etc. A patch, small and lightweight and easily counted, preferably in a sealed disposable tamper proof wrapper, serves as a convenient vehicle to manufacture, sell and distribute a medicine, medication or other product to be inhaled by a patient.

The appearance of the patch can be plain or decorated. Decorations could be graphic animals, designs, colors, logos, characters, or even identification of the medicine, medication or product.

The shape of the patch can be circular, oval, rectangular, square, triangular, or any number of sides (e.g. pentagon, hexagon, octagon, star, cross, lightning bolt, etc.), whether symmetrical or asymmetrical, regular or irregular.

In preferred embodiments, to the extent possible, an indicator can be configured to demonstrate that the patch has sufficient dosage remaining, is expired, or other signal/monitoring functions. Such an indicator can change color, or become transparent or opaque, or other indication means.

The patch preferably can be configured for any age, size or weight person.

Various materials can be used for the components of embodiments of the disclosed invention. In particular, in preferred embodiments a plurality of layers is used to perform different functions.

First, an adhesive layer or adhesion layer is preferably used to attach the patch to a face mask. The first adhesion layer can comprise of any suitable material such as plastic, latex/rubber, vinyl, acrylic, fabric or other suitable material that can adhere or have an adhesive material capable of adhering to a facial mask or facial covering.

It is further encouraged that the adhesion layer has holes to encourage communication of airspace between the front and back sides of the patch.

It is also preferable to place a disposable cover over the adhesion layer or adhesive material to protect the adhesive material from being contaminated during manufacture, distribution and sale—prior to use of the patch. The disposable cover can comprise wax paper, plastic film or other material that is easy to peel away and remove from the adhesion layer immediately prior to applying the patch to a facial mask or covering.

A second permeable layer is preferable to be utilized as a vehicle for storage and delivery of the inhalable product to the airspace proximate to the patch. Gauze, fabric or other breathable materials are suitable for use on this layer.

Similar to a disposable cover to cover the adhesive, a disposable seal is also preferable to place over the permeable layer to protect the inhalable product from being contaminated or diluted during manufacture, distribution and sale—prior to use of the patch. The disposable seal can also comprise wax paper, plastic film or other material that is easy to peel away and remove from the permeable layer immediately prior to using the patch to a facial mask or covering.

In some embodiments, particularly where the adhesive material is on the same face or side of the patch as the permeable layer, a single disposable cover can also function to cover both the adhesive material as well as the permeable layer. In such embodiments, a single disposable cover acts as a disposable cover as well as a disposable seal on one side of the patch. However, it may be helpful to also have a disposable seal on the opposite side of the patch to cover any holes in the adhesion layer.

Embodiments of the invention, including the adherable patches in the various figures (described later in the present disclosure), are preferably fully encapsulated in a disposable tamper-proof wrapper or coating, sealed bag, sealed package, envelope, or other protective airtight vehicle to protect the patch and inhalable product therein to be protected from contamination, dilution, or other environmental conditions. It is also preferable that such a disposable wrapper is either transparent or translucent so that the patch or any writing on the patch can be readable.

### Methods & Systems

Methods according to the above apparatus are also disclosed. Namely, without limitation, one such method consists of:

providing an adherable patch impregnated with the inhalable product;

removing a disposable tamper proof wrapper from the adherable patch;

removing a disposable cover, if configured, from the adherable patch exposing an adhesive material for configuring the patch to a facial mask;

attaching the patch to the facial mask, either to the outside or inside of the facial mask; and,

removing a disposable seal, if configured, thereby exposing the inhalable product; and,

configuring the facial mask over a human respiratory airway.

Other embodiments of methods may incorporate previously discussed features such as a removal of a disposable cover, removal of a disposable seal, removal of a disposable tamper proof wrapper, or embodiments where the inhalable product is a medication prescribed by a physician.

### Systems

Systems according to the above apparatus are also disclosed. Namely, without limitation, one such system consists of:

a facial mask providing communication of airspace proximate to a human respiratory airway; and,

an adherable patch for attachment to the facial mask, wherein the patch is impregnated with an inhalable product, wherein the patch delivers a desired quantity of the inhalable product into the airspace proximate to the human respiratory airway.

Other embodiments of systems may incorporate previously discussed features such as a disposable cover, a disposable seal, a disposable tamper proof wrapper or embodiments where the inhalable product is a medication prescribed by a physician.

### Discussion of the Figures

### (Patch Worn Outside of Mask)

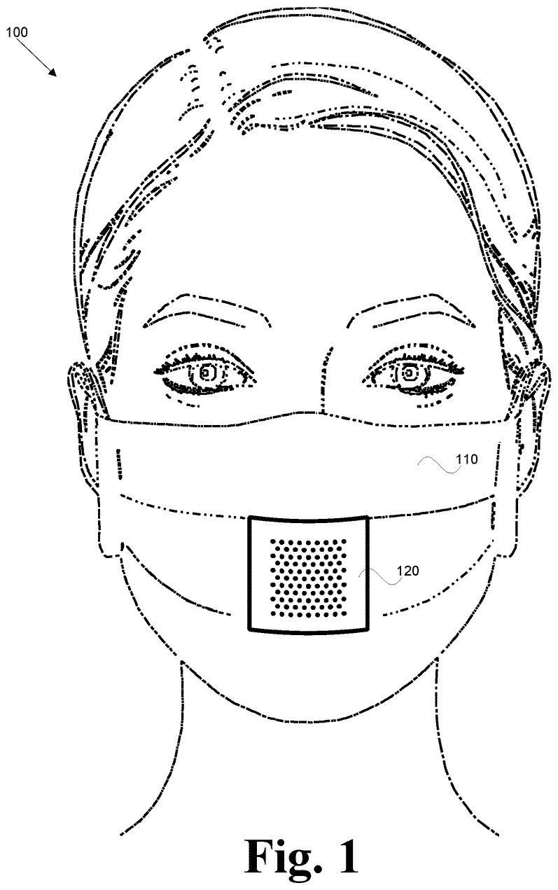

FIG. 1 is an environmental front view of an embodiment of an adherable, perforated patch 120 applied to the outside of a facial mask 110 worn by a human 100.

Although the patch 120 is centered on the facial mask 110, it may be configured further left or right of the center of the facial mask 110, or higher or lower than that illustrated. It is preferable that the patch 120 be situated in close proximity to the nose and mouth of the human 100.

Similarly, the patch 120 as illustrated is square in shape, but the patch 120 may also be configured in other embodiments as any shape, scale and thickness as desired for a particular application. Although the patch 120 is illustrated as being attached to the facial mask 110 on the outside in this embodiment, other embodiments exist (as disclosed in other figures) where the patch 120 may be configured inside the facial mask 110.

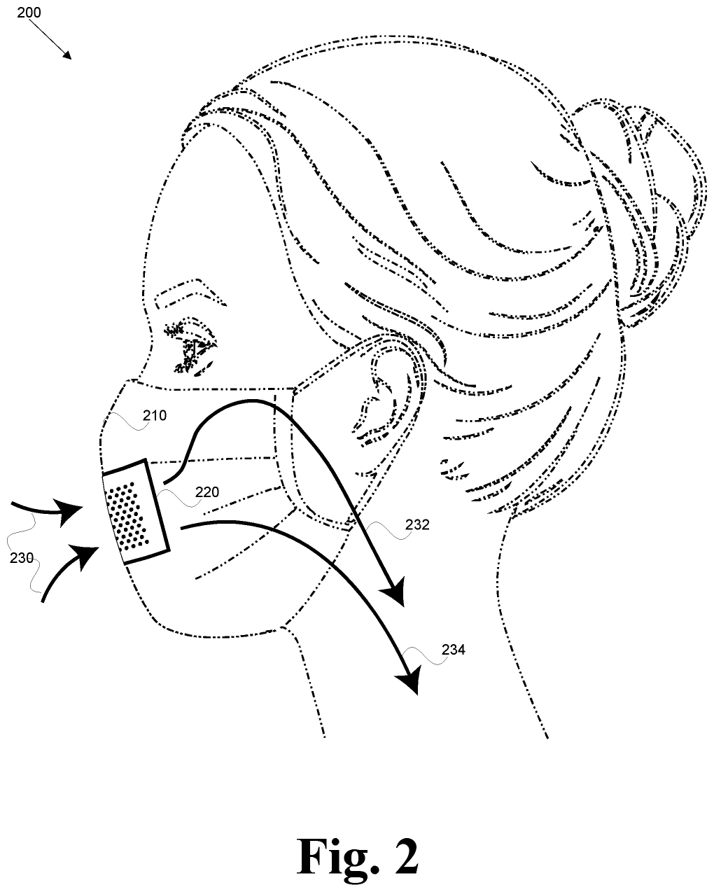

FIG. 2 is an environmental side view of FIG. 1. More particularly, a human 200 is illustrated wearing a facial mask 210 with an adherable patch 220.

Fresh air 230 is shown passing through the patch 220 and being inhaled into proximate respiratory airways (e.g. nose and mouth, not illustrated as they are behind the mask). The fresh air 230 becomes upper inhaled air 232 that passes through the nose respiratory airway (not illustrated) and lower inhaled air 234 that passes through the mouth respiratory airway (not illustrated). Together air 232 and air 234 comprise a desired quantity of inhalable product (not illustrated) from the patch 220.

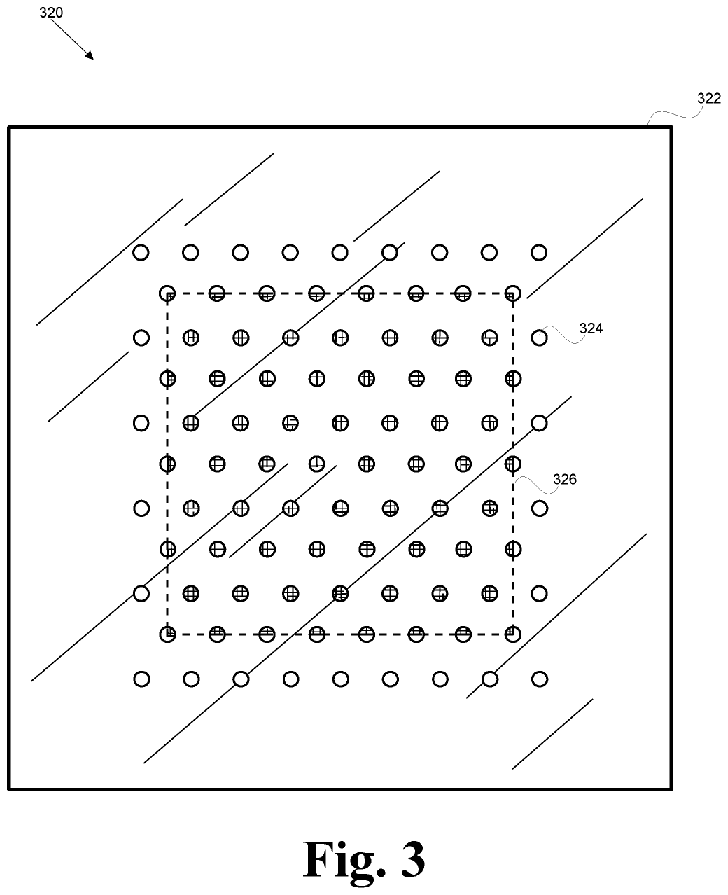

FIG. 3 is a closer front view of the embodiment of an adherable, perforated patch in FIGS. 1 & 2. More particularly, an adherable perforated patch 320 is shown, comprising an adhesion layer 322 with perforations or holes 324, as well as a permeable layer 326 which is visible only through the holes 324.

The adhesion layer 322 may comprise any shape, scale, thickness and material as desired or needed for a particular application. In the illustration the holes 324 improve circulation of air through the adhesion layer 322, allowing air to more effectively communicate between the front and back sides of the adhesion layer 322. In particular, as illustrated in FIG. 2, when the patch is attached to the outside of the facial mask 210, fresh air 230 is necessary to pass through the patch 320 to become inhaled air 232 and 234, via the holes 324. Alternatively, the adhesion layer 322 may also be breathable and allow communication of air between the front and back sides of the patch 320.

The permeable layer 326 is shown in as an outline and through the holes 324 since the permeable layer 326 is behind the adhesion layer 322 in the illustration. The permeable layer 326 is intended to contain and deliver the inhalable product (not shown as it is impregnated in the permeable layer) to proximate airspace (not illustrated) to the patch 320.

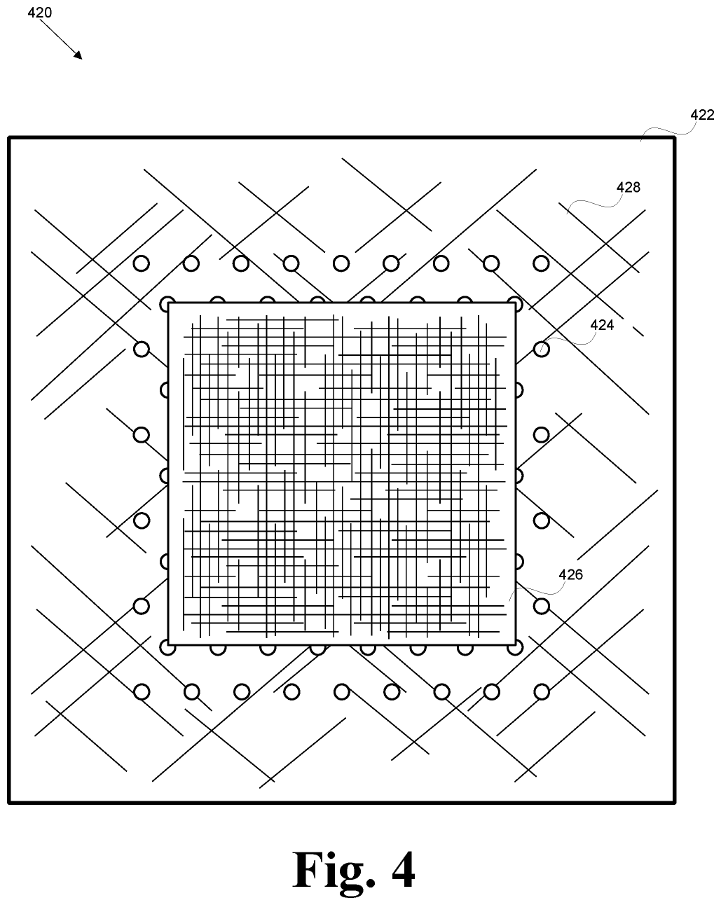

FIG. 4 is a back view of the embodiment of an adherable, perforated patch in FIGS. 1-3. More particularly, a patch 420 similar to the reverse side of patch 320 of FIG. 3 is illustrated.

The adhesion layer 422 further comprises an adhesive material 428 to attach the patch 420 to the outside of a facial mask (not shown). A permeable layer 426 impregnated with inhalable product (not shown) is similar to the permeable layer 326 of FIG. 3. The holes 324 from FIG. 3 are illustrated as holes 424 in FIG. 4, allowing communication of air between the respective sides of the adhesion layer 422.

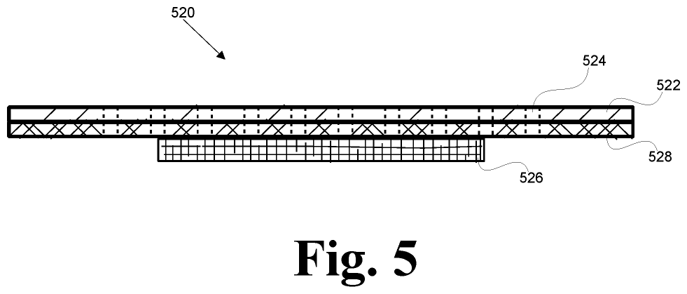

FIG. 5 is a cross-section side view of the embodiment of an adherable patch 520 similar to patches in FIGS. 1-4. More particularly, the adherable, perforated patch 520 is illustrated, comprising of an adhesion layer 522, an adhesive material 528 and a permeable layer 526, similar to the adhesion layer 422, the adhesive material 428 and permeable layer 426 illustrated in FIG. 4, respectively.

The adhesion layer 522 is preferably configured with holes 524 that encourage communication of airspace between the respective sides of the adhesion layer 522, namely allowing inhaled air to pass through the adhesion layer 522.

### Discussion of the Figures

### (Patch Worn Inside of Mask)

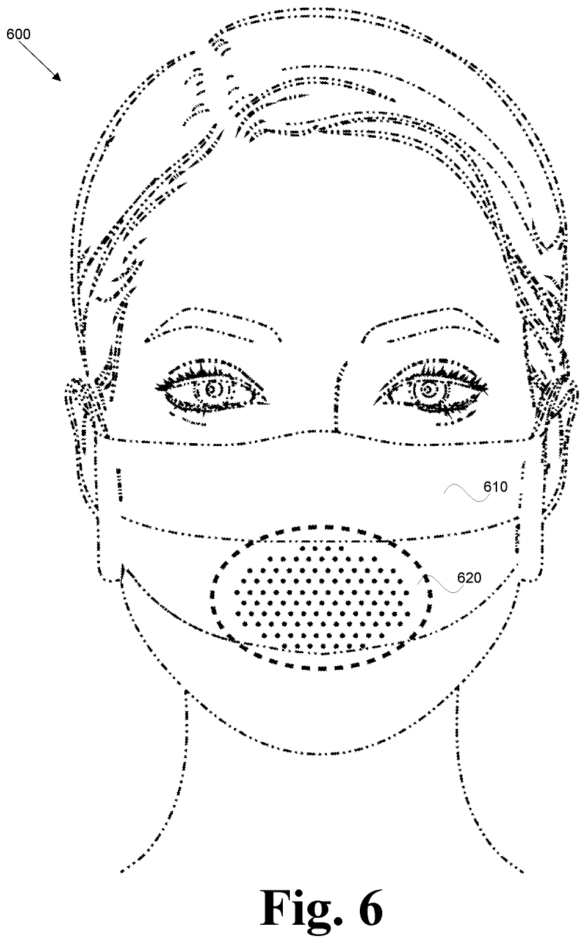

FIG. 6 is an environmental front view of an embodiment of an adherable, perforated patch 620 applied to the inside of a facial mask 610 worn by a human 600.

The patch 120 is shown in dashed line as an outline as it is adjacent and behind mask 610 as it is being worn in the inside of mask 610. Similar with FIG. 1, it is preferable that the patch 620 be situated in close proximity to the nose and mouth of the human 600.

Similarly, the patch 620 as illustrated is oval in shape, but the patch 620 may also be configured in other embodiments as any shape, scale and thickness as desired for a particular application.

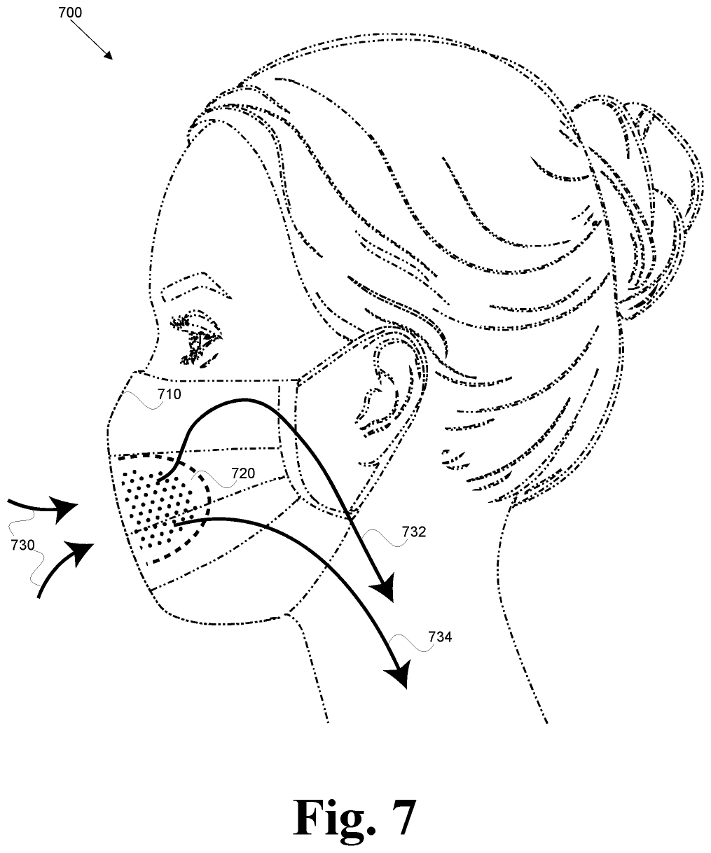

FIG. 7 is an environmental side view of FIG. 6. More particularly, a human 700 is illustrated wearing a facial mask 710 with an adherable patch 720.

In contrast to FIG. 2, where the patch 220 is worn outside the mask 210, in the present FIG. 7, the patch 720 is worn inside the mask 710. Fresh air 730 is shown passing through the patch 720 and being inhaled into proximate respiratory airways (e.g. nose and mouth, not illustrated as they are behind the mask). The fresh air 730 becomes upper inhaled air 732 that passes through the nose respiratory airway (not illustrated) and lower inhaled air 734 that passes through the mouth respiratory airway (not illustrated). Together air 732 and air 734 comprise a desired quantity of inhalable product (not illustrated) emanating from the patch 720.

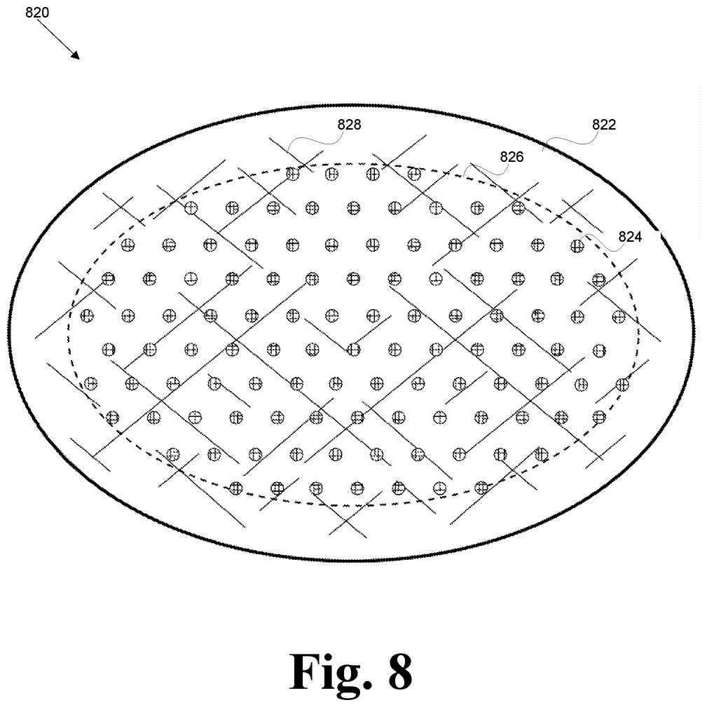

FIG. 8 is a front view of the embodiment of an adherable, perforated patch 820 similar to patches in FIGS. 6 & 7. More particularly, the patch 820 is shown, comprising an adhesion layer 822 with perforations or holes 824, as well as a permeable layer 826 which is visible only through the holes 824. Since the patch 820 as illustrated is worn on the inside of a facial mask (not shown), an adhesive material 828 is configured on the adhesion layer 822, such that the visible side of the patch 820 would be applied to the inside of the facial mask (not shown).

The adhesion layer 822 may comprise any shape, scale, thickness and material as desired or needed for a particular application. In the illustration the holes 824 improve circulation of air through the adhesion layer 822, allowing air to more effectively communicate between the front and back sides of the adhesion layer 822. In particular, as illustrated in FIG. 7, when the patch 720 is attached to the inside of the facial mask 710, fresh air 730 is necessary to pass through the patch 720 to become inhaled air 732 and 734, via the holes 724 (holes 824 in this figure). Alternatively, the adhesion layer 822 may also be breathable and allow communication of air between the front and back sides of the patch 820.

The permeable layer 826 is shown in as an outline and through the holes 824 since the permeable layer 826 is behind the adhesion layer 822 in the illustration. The permeable layer 826 is intended to contain and deliver the inhalable product (not shown as it is impregnated in the permeable layer) to proximate airspace (not illustrated) to the patch 820.

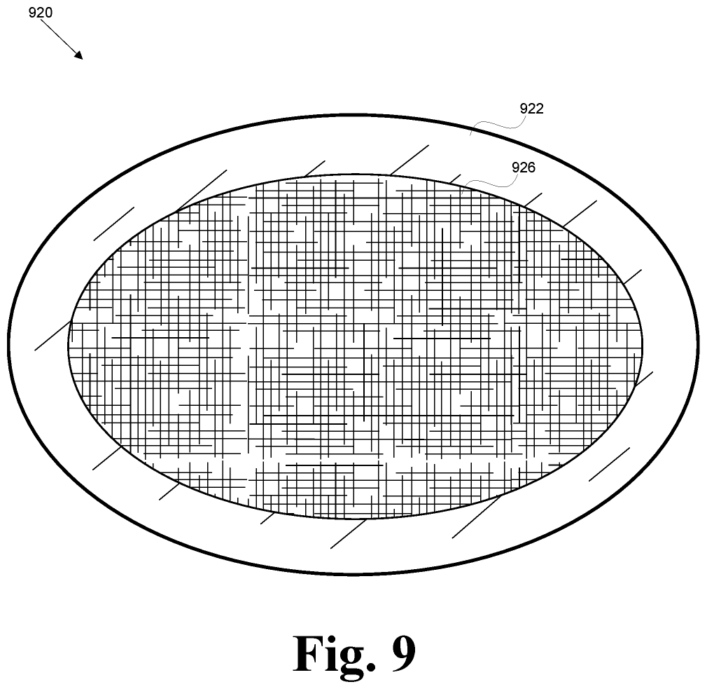

FIG. 9 is a back view of the embodiment of an adherable, perforated patch in FIGS. 6-8. More particularly, a patch 920 similar to the reverse side of patch 820 of FIG. 8 is illustrated.

A permeable layer 926 impregnated with inhalable product (not shown) is configured upon an adhesion layer 922. In the present illustration, holes (if present at all) in the adhesion layer 922 are not shown as they are entirely blocked from view by the permeable layer 926.

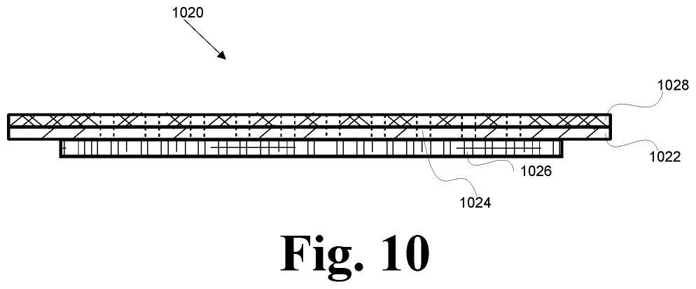

FIG. 10 is a cross-section side view of the embodiment of an adherable patch 1020 similar to patches in FIGS. 6-9. More particularly, an adherable patch 1020 is illustrated, comprising of an adhesion layer 1022, an adhesive material 1028 and a permeable layer 1026, similar to the adhesion layer 822, the adhesive material 828 and permeable layer 826 illustrated in FIG. 8, respectively.

The adhesion layer 1022 is preferably configured with holes 1024 that encourage communication of airspace between the respective sides of the adhesion layer 1022, namely allowing inhaled air to pass through the adhesion layer 1022.

### Discussion of the Figures

### (Disposable Cover & Seal)

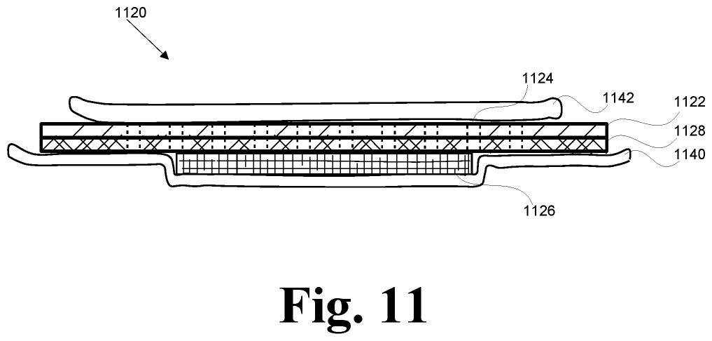

FIG. 11 is a cross-section side view of the embodiment of an adherable, perforated patch 1120 intended to be worn on the outside of a facial mask (not shown), configured with a disposable cover 1140 and a disposable seal 1142. Similar to FIG. 5, the patch 1120 comprises an adhesion layer 1122, an adhesive material 1128 and a permeable layer 1126.

The adhesion layer 1122 is preferably configured with holes 1124 that encourage communication of airspace between the respective sides of the adhesion layer 1122, namely allowing inhaled air to pass through the adhesion layer 1122.

The disposable cover 1140 in usage can be removed from the patch 1120, thereby exposing both the adhesive material 1128 and the permeable layer 1126. As noted earlier in the disclosure, the disposable cover acts to protect both the adhesive material 1128 and permeable layer 1126 from contamination and dilution until use of the patch 1120. It can be manufactured from any disposable material, such as wax paper or plastic film, or other materials suitable as a temporary protectant.

The disposable seal 1142 can also be used to protect the inhalable product (not shown) impregnated in the permeable layer 1126 by covering the holes 1124 in the adhesion layer 1122 opposite of the disposable cover 1140. Similar to the disposable cover 1140, the disposable seal 1142 can be manufactured from any disposable material, such as wax paper or plastic film, or other materials suitable as a temporary protectant or sealant.

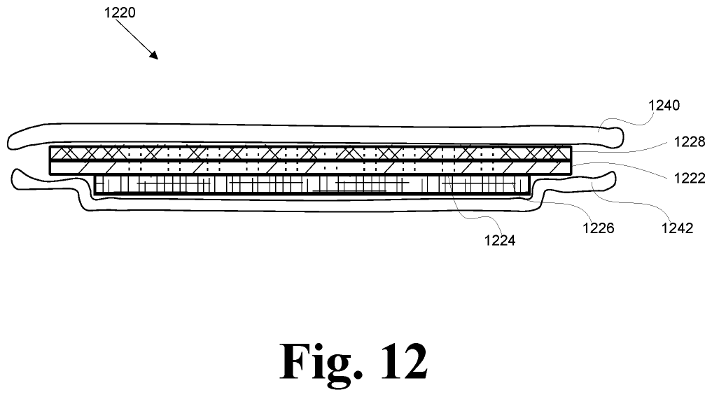

FIG. 12 is a cross-section side view of the embodiment of an adherable, perforated patch 1220 intended to be worn on the inside of a facial mask (not shown), comprising an adhesion layer 1222, a quantity of holes 1224 in the adhesion layer 1222 to encourage communication of airspace through the adhesion layer 1222, an adhesive material 1228 on the adhesion layer 1222, and a permeable layer 1226 comprising an inhalable product (not shown) impregnated into the permeable layer 1226.

In the figure, the patch 1220 may also comprise a disposable cover 1240 to protect the adhesive material 1228 and may also comprise a disposable seal 1242 to protect the inhalable product (not shown) impregnated in the permeable layer 1226.

Similar to the disposable cover 1140 in FIG. 11, the disposable cover 1240 in FIG. 12 usage can be removed from the patch 1220, thereby exposing the adhesive material 1228. However, since the permeable layer 1226 is on the opposite side as the adhesive material 1228, the disposable seal 1242 similarly functions to protect the permeable layer 1226 (in particular the inhalable product impregnated in the permeable layer) from contamination and dilution. Similar to the disposable cover 1240, the disposable seal 1242 can be manufactured from any disposable material, such as wax paper or plastic film, or other materials suitable as a temporary protectant or sealant.

### Discussion of the Figures

### (Methods)

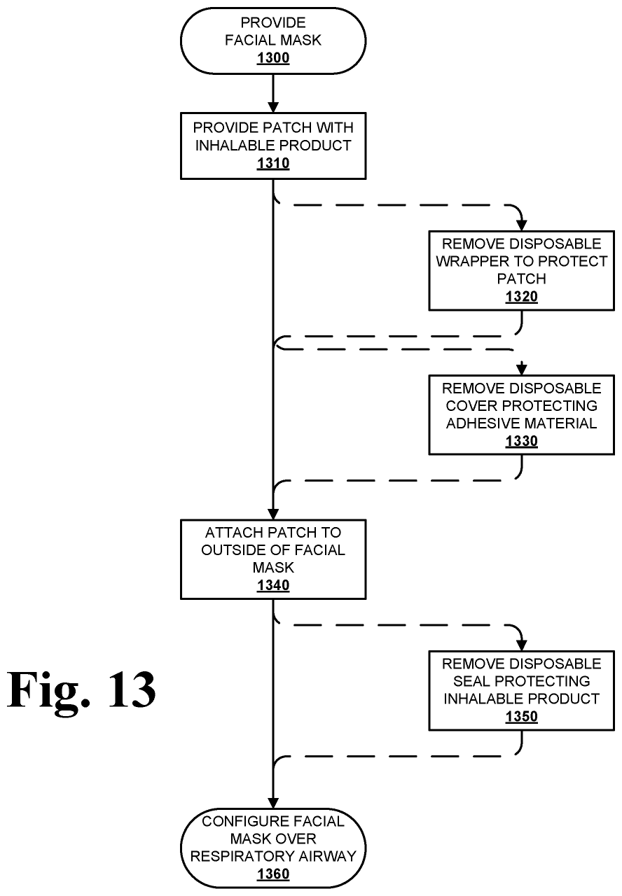

FIG. 13 is a flow chart of an embodiment of a method of use of an adherable patch worn on the outside of a facial mask.

The process commences with providing a facial mask for use in step 1300.

In step 1310, an adherable patch comprising an inhalable product described herein is provided.

In step 1320, if a disposable tamper proof wrapper is provided on the adherable patch, the wrapper is removed, thereby making available the adherable patch to be attached to the facial mask.

In step 1330, if a disposable adhesive cover is provided on the adherable patch to protect an adhesive material, the cover is removed thereby exposing the adhesive material.

In step 1340, the adherable patch is configured onto the outside of the facial mask utilizing the adhesive material exposed by the removal of the disposable cover.

In step 1350, if a disposable adhesive seal is provided on the adherable patch to protect the inhalable product, the seal is removed thereby exposing the inhalable product.

The process is completed in step 1360 with the user configuring the facial mask (now with the patch configured on the outside), over their nose and mouth (e.g. covering their respiratory airways).

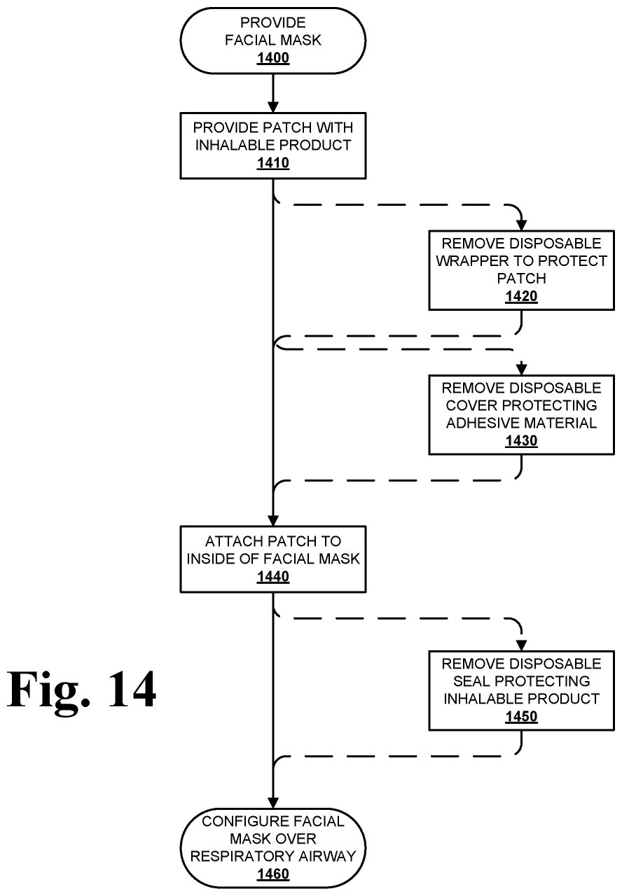

FIG. 14 is a flow chart of an embodiment of a method of use of an adherable patch worn on the inside of a facial mask.

The process commences with providing a facial mask for use in step 1400.

In step 1410, an adherable patch comprising an inhalable product described herein is provided.

In step 1420, if a disposable tamper proof wrapper is provided on the adherable patch, the wrapper is removed, thereby making available the adherable patch to be attached to the facial mask.

In step 1430, if a disposable adhesive cover is provided on the adherable patch to protect an adhesive material, the cover is removed thereby exposing the adhesive material.

In step 1440, the adherable patch is configured onto the inside of the facial mask utilizing the adhesive material exposed by the removal of the disposable cover. As noted earlier in the disclosure, other methods of adhesion (e.g. hook-loop fabric such as Velcro) or placement (e.g. pocket) can be used with equal success.

In step 1450, if a disposable adhesive seal is provided on the adherable patch to protect the inhalable product, the seal is removed thereby exposing the inhalable product.

The process is completed in step 1460 with the user configuring the facial mask (now with the patch configured on the outside), over their nose and mouth (e.g. covering their respiratory airways).

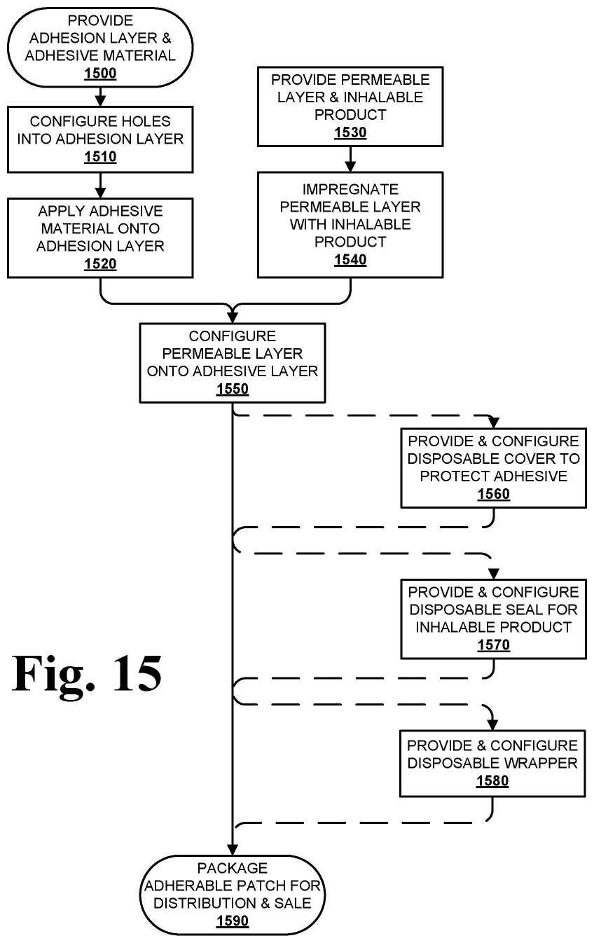

FIG. 15 is a flow chart of an embodiment of a method of manufacture of an adherable patch.

The process commences with providing an adhesion layer and adhesive material in step 1500.

In step 1510, holes can be configured into the adhesion layer to promote communication or exchange of air through the adhesion layer.

The adhesive material is applied to the adhesion layer in step 1520, providing a reliable result when configuring a patch onto a facial mask. It is noted that although all steps of the methods can be in a different order than illustrated, in particular steps 1510 and 1520 can be switched or performed concurrently since it may depend on the particular application whether to configure holes or apply adhesion material first.

In step 1530, the manufacture of the permeable layer is commenced. It is noted that the manufacture of the permeable layer can be performed in parallel with the manufacture of the adhesion layer, such that steps 1500 through 1540 can all be performed and ordered interchangeably depending upon the embodiment or application. More particularly, in step 1530 a permeable layer and an inhalable product are provided.

In step 1540, the inhalable product is impregnated into the permeable layer.

Having now manufactured the adhesion layer and the permeable layer in steps 1500 through 1540, the layers are combined in step 1550. More particularly, the permeable layer is configured onto the adhesion layer. This can be accomplished in a number of ways, but if adhesive material is on the same side of the adhesion layer, said adhesion material can be used to also configure the permeable layer onto the adhesion layer.

In step 1560, if desirable a disposable cover can be provided and configured to protect the adhesive material on the adhesion layer (e.g. FIGS. 11 & 12). If the permeable layer is on the same side of the adhesion layer as the adhesive material, then the disposable cover protecting the adhesive material can also be configured to protect the permeable layer (e.g. FIG. 11).

In step 1570, if desirable a disposable seal can be provided and configured to seal the permeable layer (e.g. FIG. 12) and/or holes (e.g. FIG. 11) through the adhesive layer to protect the inhalable product.

In step 1580, it is preferable that the entire adherable patch be encapsulated in a disposable, tamper proof wrapper to protect the various components and inhalable product of the adherable patch.

The process is completed in step 1590, when the adherable patch is further labeled, packaged or made ready for distribution and sale.

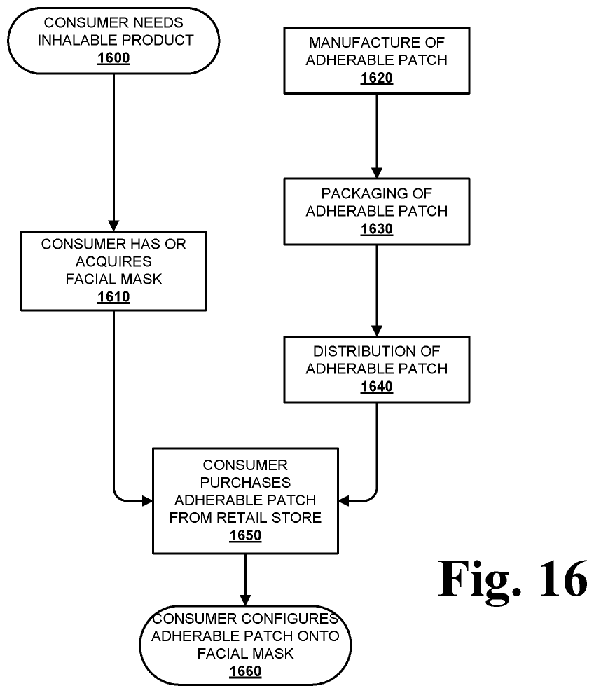

FIG. 16 is a flow chart of an embodiment of a method of procurement of an adherable patch.

The process is commenced in step 1600 when a consumer is in need of an inhalable product, such as an essential oil or non-prescribed substance (e.g. over the counter product).

In step 1610, the consumer has or acquires a facial mask, which may be consistent with needing a inhalable product (e.g. respiratory problems so consumer wears a mask to prevent infection of self/others).

In step 1620, an adherable patch is manufactured (e.g. steps of FIG. 15), with the patch packaged in step 1630, and distributed (e.g. wholesale channels, etc.) in step 1640.

In step 1650, the consumer purchases the adherable patch from a retail store (e.g. over the counter, grocery store, health food store, etc.).

The process is completed in step 1660, wherein the consumer configures the adherable patch onto the facial mask (e.g. see FIGS. 13 & 14).

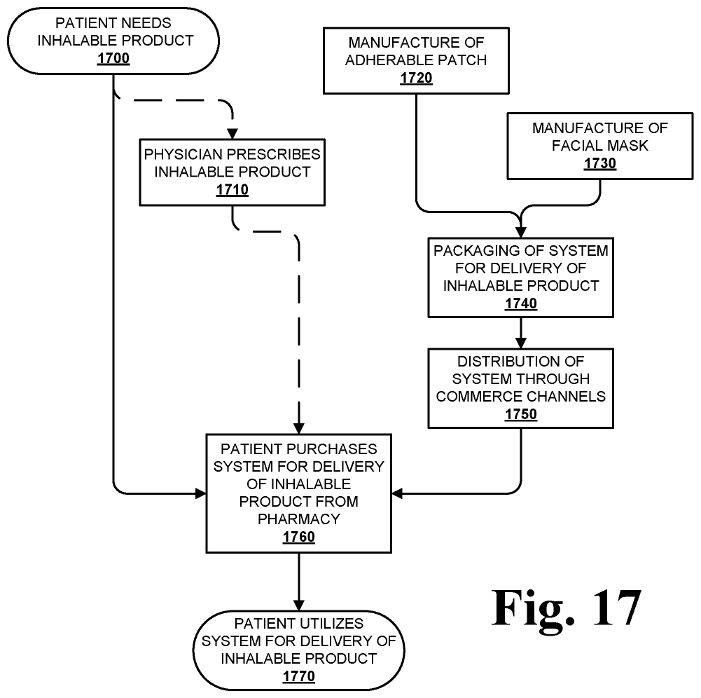

FIG. 17 is a flow chart of an embodiment of a method of procurement of a system for delivery of an inhalable product. This figure is similar in many respects to FIG. 16, with the exceptions that the consumer is a patient, the inhalable product is a prescribed medication, and the embodiment is packaged as a system for delivering an inhalable product (e.g. an adherable patch with a corresponding facial mask).

The process is commenced in step 1700 when a patient is in need of an inhalable product, such as a physician prescribed medication (e.g. steroid, antibiotic, anesthetic, etc.).

In step 1710, the patient's physician prescribes the medication and the prescription is sent to the patient's pharmacy.

In step 1720 an adherable patch is manufactured (e.g. steps of FIG. 15), and in step 1730 a facial mask is manufactured.

The adherable patch and facial mask are then packaged together creating a system for delivery of an inhalable product in step 1740. The system is distributed (e.g. wholesale channels for controlled substances, etc.) in step 1750.

In step 1760, the patient purchases the system for delivery of an inhalable product from the pharmacy where the physician's prescription was received.

The process is completed in step 1770, wherein the patient configures the adherable patch onto the facial mask (e.g. see FIGS. 13 & 14).

### Applications

Embodiments of the present invention may be used in a variety of contexts. Inhalable products may be medications (e.g. prescribed by a physician), or over the counter products (e.g. essential oils, non-medication inhaled supplements, etc.).

Apparatus and system embodiments may be labeled, packaged, distributed and used independently as an adherable patch, or may be combined with a facial mask to be packaged, distributed and used as a system. Method embodiments may comprise usage of the adherable patch, manufacture of the adherable patch, as well as methods more directed toward the packaging and distribution of the patch or system.

In usage, the patch or the system may be used in the home, or may be used in care center environments (e.g. senior/assisted living accommodations), or may be used in medical settings (e.g. hospitals, urgent care centers, etc.), as well as a variety of other medical and non-medical contexts. One of the benefits of the patch and the system is that the inhalable product may be administered without the need for power or electricity (e.g. nebulizer) or compressed gas (e.g. an inhaler comprising a container with compressed gas). It is further noted that embodiments of the invention do not require needles or any invasive procedure (e.g. needle prick) to deliver a dosage of a medication or product.

### Conclusion

There are numerous and diverse additional embodiments anticipated by the present invention. Therefore, it is further understood that the following examples are not limiting by nature, but rather alternate specific examples where the disclosed apparatus and method can be utilized.

The novel and useful approaches described herein evidence a variety of benefits over prior art approaches. In particular, embodiments of the present invention provide one or more additional aspects of enhanced convenience, usability and reliability over the prior art.

Preferred embodiments of this invention are described herein, including the best mode known to the inventor for carrying out the invention. As can be appreciated from the technical disclosure herein, embodiments of the present invention evidence a variety of advances and benefits over the prior art, uniquely and advantageously yielding savings of time, effort, and cost relative to the existing prior art currently utilized. Of course, variations on those preferred embodiments will become apparent to those of ordinary skill in the art upon reading the foregoing description. The inventor expects skilled artisans to employ such variations as appropriate, and the inventor intends for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention. Groupings of alternative elements or embodiments of the invention disclosed herein are not to be construed as limitations. Each group member may be referred to and claimed individually or in any combination with other members of the group or other elements found herein.

Furthermore, if any references have been made to patents and printed publications in this specification, then each of the above cited references and printed publications, if any, are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### Literal Recitation

The following paragraphs are included to provide literal recitation of the claims, and in no way limit or constrain future claims that may be made or amended.

An adherable patch comprising an inhalable product, the patch designed for being applied to a facial mask and delivering a quantity of the inhalable product to a human respiratory airway, is disclosed. The adherable patch may comprise of a plurality of layers, the plurality of layers may comprise a first adhesion layer and a second permeable layer. The adhesion layer may comprise an adhesive material for attachment to the facial mask. The adherable patch may further comprise a disposable cover to protect the adhesive material prior to application of the patch to the facial mask. The adhesion layer may comprise a plurality of holes to effect communication of airspace proximate to both sides of the adhesion layer. The inhalable product may be is impregnated in the permeable layer. The adherable patch may further comprise a disposable seal configured over the permeable layer to protect the inhalable product prior to use of the patch. The adherable patch may further comprise a disposable tamper-proof wrapper encapsulating the entire patch, thereby encapsulating the adhesion layer and permeable layer. The adherable patch may further comprise a disposable cover to protect the first adhesion layer and the second permeable layer prior to use of the patch. The inhalable product may comprise a physician prescribed medication or a non-medication substance. The inhalable product may comprise respiratory medications or non-respiratory medications, such steroids, antibiotics, anesthetics, corticosteroids, beta-sympathomimetics, muscarinic antagonists, bronchodilators, mucolytics, airway surface hydrators, or non-medications such as essential oils or other non-prescribed substances. The patch may comprise a plurality of layers, the plurality of layers comprising a first adhesion layer and a second permeable layer; wherein the first adhesion layer comprises an adhesive material and functions to adhere the patch to the face mask, and further comprises a plurality of holes to effect communication of airspace proximate to both sides of the first adhesion layer. The patch may comprise a disposable cover to protect the adhesive material prior to application of the patch to the facial mask. The second permeable layer may be impregnated with the inhalable product, thereby delivering the inhalable product to airspace proximate to a human respiratory airway when the facial mask is worn. The patch may comprise a disposable seal configured over the permeable layer to protect the inhalable product prior to use of the patch.

A method for delivering a quantity of an inhalable product to a human respiratory airway is disclosed, comprising: providing an adherable patch impregnated with the inhalable product; removing a disposable tamper proof wrapper, removing a disposable cover from the adherable patch exposing an adhesive material for configuring the patch to the facial mask; attaching the patch to a facial mask; and, configuring the facial mask over the human respiratory airway. The method may further comprise removing a disposable seal over the inhalable product prior to configuring the mask over the human respiratory airway. The inhalable product in the method may be a medication prescribed by a physician or a non-medication substance. The step of attaching the patch to a facial mask may comprise attaching the patch to an outside surface of the facial mask or an inside surface of the facial mask. The method may further comprise removing a disposable seal over the inhalable product prior to configuring the mask over the human respiratory airway. The method may further comprise removing a disposable tamper proof wrapper prior to attaching the patch to a facial mask.

A system for delivering an inhalable product to a human respiratory airway is disclosed, comprising: a facial mask providing communication of airspace proximate to the human respiratory airway; and, an adherable patch for attachment to the facial mask, wherein the patch is impregnated with the inhalable product, wherein the patch delivers a desired quantity of inhalable product into the airspace proximate to the human respiratory airway. The facial mask may be distributed in the same packaging as the adherable patch. The patch may comprise a plurality of layers, the plurality of layers comprising a first adhesion layer and a second permeable layer. The first adhesion layer may comprise an adhesive material and functions to adhere the patch to the face mask. The patch in the system may further comprise a plurality of holes to encourage communication of airspace proximate to both sides of the first adhesion layer. The patch in the system may further comprise a disposable cover to protect the first adhesive material prior to application of the adherable patch to the facial mask. The second permeable layer may be impregnated with the inhalable product, thereby delivering a desired quantity of the inhalable product to airspace proximate to a human respiratory airway when the facial mask is worn. The patch in the system may comprise a disposable seal configured over the permeable layer to protect the inhalable product prior to use of the adherable patch.

1. An adherable patch comprising an inhalable product, the patch comprising a first adhesion layer for attachment, and applied to a separate facial mask, and delivering a quantity of the inhalable product to a human respiratory airway.

2. The adherable patch of claim 1, wherein the patch comprises of a plurality of layers, the plurality of layers comprising the first adhesion layer and a second permeable layer.

3. The adherable patch of claim 2, the first adhesion layer comprising an adhesive material for attachment to the facial mask.

4. The adherable patch of claim 3, further comprising a disposable cover to protect the adhesive material prior to application of the patch to the facial mask.

5. The adherable patch of claim 2, wherein the first adhesion layer comprises a plurality of holes to effect communication of airspace proximate to both sides of the first adhesion layer.

6. The adherable patch of claim 2, wherein the inhalable product is impregnated in the second permeable layer.

7. The adherable patch of claim 6, further comprising a disposable seal configured over the second permeable layer to protect the inhalable product prior to use of the patch.

8. The adherable patch of claim 2, further comprising a disposable tamper-proof wrapper encapsulating the first adhesion layer and the second permeable layer of the adherable patch, but not encapsulating the facial mask.

9. The adherable patch of claim 1, wherein the inhalable product is a prescription drug.

10. The adherable patch of claim 9, wherein the inhalable product is a steroid.

11. The adherable patch of claim 9, wherein the inhalable product is an antibiotic.

12. The adherable patch of claim 9, wherein the inhalable product is an anesthetic.

13. The adherable patch of claim 1:
wherein the patch comprises a plurality of layers, the plurality of layers comprising a first adhesion layer and a second permeable layer;
wherein the first adhesion layer comprises an adhesive material and functions to adhere the patch to the face mask, and further comprises a plurality of holes to effect communication of airspace proximate to both sides of the first adhesion layer;
wherein the second permeable layer is impregnated with the inhalable product, thereby delivering the inhalable product to airspace proximate to the human respiratory airway when the facial mask is worn;
wherein the inhalable product is a prescription drug;
further comprising a disposable cover to protect the adhesive material prior to application of the patch to the facial mask;
further comprising a disposable seal configured over the second permeable layer to protect the inhalable product prior to use of the patch; and,
further comprising a disposable tamper-proof wrapper encapsulating the first adhesion layer and the second permeable layer.

14. A method for delivering a quantity of an inhalable product to a human respiratory airway, comprising:
providing an adherable patch impregnated with the inhalable product;
removing a disposable cover from the adherable patch exposing an adhesive material for configuring the patch to a separate facial mask;
attaching the patch to the facial mask; and,
configuring the facial mask over the human respiratory airway.

15. The method of claim 14, further comprising removing a disposable tamper proof wrapper encapsulating the patch only, but not the separate facial mask.

16. The method of claim 14, wherein the inhalable product is a medication prescribed by a physician.

17. The method of claim 14, wherein the step of attaching the patch to a facial mask comprises attaching the patch to an outside surface of the facial mask.

18. The method of claim 14,
further comprising removing a disposable tamper proof wrapper prior to attaching the patch to the facial mask;
further comprising removing a disposable seal over the inhalable product prior to configuring the mask over the human respiratory airway;
wherein the inhalable product is a medication prescribed by a physician; and,
wherein the step of attaching the patch to a facial mask comprises attaching the patch to an inside surface of the facial mask.

19. (canceled)

20. (canceled)
